(19) United States
(12) Patent Application Publication
(10) Pub. No.: US 2002/0160025 A1
(43) Pub. Date: Oct. 31, 2002

(54) Cosmetic preparations for skin and hair care

(76) Inventor: Marina Mustapic, Zagreb (HR)

Correspondence Address: Fitch Even Tabin & Flannery, 120 South LaSalle Street Suite 1600, Chicago, IL, 60603-3406, US

(21) Appl. No.: 09/936,825

(22) PCT Filed: Jan. 17, 2001
(86) PCT No.: PCT/HR01/00001

(30) Foreign Application Priority Data
Jan. 18, 2000 (HR) P20000029A

(51) Int. Cl. A61K 7/06

(52) U.S. Cl. 424/401

(57) ABSTRACT

Cosmetic preparations for skin and hair care are manufactured from natural plant ingredients. Every single preparation is a mixture of appropriate kinds and quantities of substances. The basic substance is zeolit, the common ones are kuzu root and ginger. These cosmetic preparations are intended for care of all types of skin and hair. Natural plant ingredients and adequate manufacturing procedure secure maintenance of all the healing properties. Executed tests show that all of the cosmetic preparations are in concordance with legislation in the field of Common use articles. Laboratory examinations and applications have so far showed that not a single preparation causes allergic reactions known up to present, nor any other side effects/contra indications.

### DESCRIPTION OF THE INVENTION

1. The Field that the Invention Refers to

The invention refers to products for medical, dental and hygienic purposes, precisely, to cosmetic and similar hygienic products for skin and hair care. According to the International patent classification (7^{th} edition) this invention is placed in section A—Human necessities A 61 K 7/00 Cosmetics or similar toilet preparations

Technical Problem

With the cosmetic products as per this invention, the entire skin and hair care is achieved by the use of natural substances which do not cause allergic reactions or other side-effects/contra indications as known up to the present, and which are absolutely independent on the type of skin or hair.

2. Background Art

Such or similar patent protected cosmetic preparations are unknown to the applicant/inventor.

### DISCLOSURE OF THE INVENTION ESSENCE

Cosmetic preparations for skin and hair care according to the invention are products in the form of creams, (e.g. day or night creams, . . . ), milks (e.g. body milk), oily emulsions (e.g. oily body emulsion), protective emulsions (e.g. sun protective emulsion), shampoos (e.g. hair shampoo).

The basic ingredient to all products is zeolit. Common ingredients to all products are kuzu root and ginger. Dependent on kind and purpose, other ingredients to the particular preparations are:

a) almond oil,

b) jojoba oil,

c) marigold (Calendula Officinalis),

d) propolis,

e) avocado oil,

f) gavez,

g) olive oil,

h) cantaraon (Hypericum Perforatum),

i) cocoa butter,

j) yarrow (Achillea Millefolium),

k) walnut,

l) beeswax,

m) water,

n) plant oil extracts and other less essential ingredients.

### DESCRIPTION OF THE PREFERRED EMBODIMENT

The substances in appropriate form (powder, oil, . . . ) are heated separately and/or as an adequate mixture to a certain temperature depending on the kind of the product. This is followed by the process of the substances and/or mixture consolidation and mixing in the course of cooling.

Adequately treated zeolit is added into the cooled mixture and the process of product finalization is ended by this.

The basic substance to all preparations is zeolit in the quantity of 0.1-70% wt, more preferably 3-50% wt, and most preferably 5-25% wt.

The common substance to all preparations is kuzu root in the quantity of 0.5-50% wt, more preferably 1-20% wt, and most preferably 1.5-2% wt.

The second common substance to all products is ginger in the quantity of 0.2-30% wt, more preferably 0.3-10% wt, and most preferably 0.5-8% wt.

The Invention Application

The cosmetic preparations as per invention are applicable to all types of skin and hair according to the specific application for each particular product All cosmetic preparations have undergone the statutory regulated tests and are provided with analytic reports issued by authorized institutions.

All the substances being of natural origin and their essential properties being not reduced and/or changed during the process of manufacturing the patented preparations do not cause allergic reactions known up to present, nor any other side effects/contra indications.

1. Use of zeolit in cosmetic preparations for skin and hair care, wherein zeolit is the basic substance to all preparations in the quantity of 0.1-70% wt, more preferably 3-50% wt, and most preferably 5-25% wt.

2. Use od kuzu root in cosmetic preparations for skin and hair care according to claim 1, wherein kuzu root is the first common substance to all preparations in the quantity of 0.5-50% wt, more preferably 1-20% wt, and most preferably 1.5-2% wt.

3. Use of ginger in cosmetic preparations for skin and hair care according to the claims 1 and 2, wherein ginger is the second common substance to all preparations in the quantity of 0.2-30% wt, more preferably 0.3-10% wt and most preferably 0.5-8% wt.

4. Use of cosmetic preparations for skin and hair care according to the claims 1-3, wherein they are intended for care of all types of skin and hair.

5. Use of cosmetic preparations for skin and hair care according to the claims 1-4, wherein the products are in the form of creams, milks, oily emulsions, protective emulsions or shampoos.
